(19) United States
(12) Patent Application Publication
(10) Pub. No.: US 2006/0165615 A1
(43) Pub. Date: Jul. 27, 2006

(54) Non-oxidative tooth whiteners for dentifrice application

(76) Inventors: Rajesh V. Shende, Columbia, MO (US); Stephen J. Lombardo, Columbia, MO (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/285,769

(22) Filed: Nov. 22, 2005

Related U.S. Application Data
(60) Provisional application No. 60/630,229, filed on Nov. 23, 2004.

(51) Int. Cl. A61K 8/96; A61K 8/20

(52) U.S. Cl. 424/50; 424/53

(57) ABSTRACT

The present invention relates to a non-oxidative approach for removing or reducing stains, which is based on reactive modification and complexation of porphyrins. This approach was found to be suitable for removing stains from hydroxyapatite, and the whiteness that was achieved using this formulation was found to be superior as compared to the whiteness obtained by using an oxidizing solution containing 3% hydrogen peroxide.

### CROSS-REFERENCE TO RELATED APPLICATIONS

This application claims priority of U.S. provisional patent application Ser. Nos. 60/630,229, filed Nov. 23, 2004, which is incorporated by reference herein.

### BACKGROUND OF THE INVENTION

Teeth are composite biological structures. The outer layer of the crown consists of the enamel, which is a calcified structure that varies from translucent to yellow-gray in color. Underneath the enamel are the dentin, and then a central core chamber of pulp. Both the enamel and dentin layers are porous. Stains can migrate into these pores by diffusion due to the dynamic environment in the oral cavity from the secretions of the salivary glands.

In general, tooth discoloration is typically due to colored substances in the tooth structure itself (instrinsic color stains) or secondarily due to accumulated stains from dietary tannins, which are often trapped in calculus (extrinsic color stains). Calculus, which is the mineralized bacterial dental plaque on enamel surfaces, can also cause discoloration. Tooth discoloration can occur in both the enamel and dentin layers, and the apparent color of the enamel-covered crowns is in part the result of the color of the underlying dentin.

In general, discoloration is typically caused by stain materials that are from compounds having conjugated diene or porphyrin groups as part or their structure. They can be produced by mouth bacteria and can accumulate under the enamel. The stain materials are generally water insoluble and require modification to solubilize them in water.

A number of products are currently available to whiten teeth. Most of the commercially available dentifrice formulations for tooth whitening contain oxidizing compounds. These oxidizing compounds are generally peroxides that can penetrate into the pores in enamel and dentin, to remove both extrinsic and intrinsic color stains. As oxidation proceeds, teeth continually whiten. However, oxidation can pose a health concern because of degradation of proteins in the enamel and dentin. As an alternative to peroxide-based formulations, however, dentifrices containing ionic types of bleaching agents or phosphate-based chelating agents are also available.

Despite the available methods for stain removal and tooth whitening, a need still exists for alternative approaches, e.g., non-oxidative, to remove stains from teeth.

### SUMMARY OF THE INVENTION

The present invention provides a non-oxidative composition for removing or diminishing stains from teeth and/or whitening teeth. The composition comprises at least one reducing agent and optionally a complexing agent. In one embodiment, two or more reducing agents can be used in the composition. Reducing agents that are useful in practicing the invention include any suitable agent that is capable of making stain compounds on teeth water soluble.

In another aspect, the invention provides a process for preparing non-oxidative compositions for removing or diminishing stains and/or whitening teeth. In another aspect, the present invention provides a non-oxidative process for removing or diminishing stains in teeth comprising contacting teeth with an effective amount of a non-oxidative, non-abrasive composition of the invention that includes a reducing agent.

### BRIEF DESCRIPTION OF THE FIGURES

FIG. 1 illustrates stained and unstained hydroxyapatite pellets, unstained (top three), and stained with a vat or a reactive dye (lower).

FIGS. 2-4 illustrate stain removal from vat dye-stained sintered hydroxyapatite pellets.

FIGS. 5A-5C illustrate the effect of addition of Solution A to a reactive dye solution.

FIGS. 6A-6D illustrate reactive dye-stain removal with Solution A and Solution B, after addition of EDTA.

FIGS. 7A-7D illustrate the modification of coffee stains in solution.

FIG. 8 illustrates spectral data for coffee stained solutions.

FIGS. 9A-9D illustrate the modification of tea stains in solution.

FIGS. 10, and 11 illustrate spectral data for tea stained solutions.

FIGS. 12A-12B illustrate unstained and tea stained hydroxyapatite pellet samples.

FIGS. 13A-13E illustrate Tea-II stain removal using Solution C and a comparison with a peroxide solution.

FIGS. 14A-14E illustrate the extraction of color into solution from Tea-II stained pellets.

FIGS. 15A-15F illustrate the extraction of color into solution from a tea stained hydroxyapatite pellet.

FIG. 16 illustrates spectral data for unstained and tea stained hydroxyapatite pellets.

FIG. 17 illustrates spectral data for the modification of tea stains extracted into solution from hydroxyapatite pellets.

FIGS. 18 and 19 illustrate cross-sectional views of tea stained hydroxyapatite pellets and pellets after treatment with a composition of the invention.

FIG. 20A and 20B illustrate the whitening of tea stained hydroxyapatite powder.

FIGS. 21A and 21B illustrate the whitening of coffee stained hydroxyapatite powder samples.

FIGS. 22A-22E illustrate the whitening that can be achieved by using 6%-12% H₂O₂ and Formulation-I on tea-stained hydroxyl apatite powder

### DETAILED DESCRIPTION OF THE INVENTION

In the following detailed description, reference is made to the accompanying figures which form a part hereof, and in which is shown by way of illustration specific embodiments in which the invention can be practiced. These embodiments are described in sufficient detail to enable those skilled in the art to practice the invention, and it is to be understood that the embodiments can be combined, or that other embodiments can be utilized and that structural and material changes can be made without departing from the spirit and scope of the present invention. The following detailed description provides examples and the scope of the present invention as defined by the appended claims and their equivalents.

The present invention is based on the discovery that many compounds that stain teeth are water insoluble and treating these compounds with a reducing agent can make them water soluble. The water soluble compounds then can be rinsed away during normal oral care.

It should be noted that references to “an, “one, or “various” embodiments in this disclosure are not necessarily to the same embodiment, and such references contemplate more than one embodiment.

The term “whiten or whitening” or “stain removal” as used herein, means “diminishing stains,” “removing discoloration,” or “removing stains,” which are adhered to, or entrapped in materials on the surface of teeth or tooth enamel and stains within the dentin, or the pulp. Diminishing or removing stains includes preventing the build up of surface attached stains or increasing the whiteness of teeth or tooth enamel. The subject matter of the present invention presumes that the native tooth whiteness has been changed with debris which, when removed, will reveal the underlying color of the tooth.

The term “reducing agent” as used herein means a compound or composition that involves the addition of bonds to hydrogen or the loss of bonds to oxygen, e.g., the gain of electrons or decrease in oxidation number.

The term “complexing agent” as used herein means a compound or composition that interacts either temporarily or permanently with the water soluble reduced form of the stain molecule to prevent the reduced stain molecule from converting back, e.g., via oxidation, to its water insoluble form.

Non-limiting examples of reducing agents useful for practicing the invention include non-metallic reducing agents and carbon compounds. Non-limiting examples of non-metallic reducing agents useful for practicing the invention include bisulfites, hydrosulphites, hydrosulfates, sodium salts of sulphurous acid (H₂SO₃), phosphorus, phosphates, phosphites, phosphoric acid, and the like. Non-limiting examples of carbon compounds useful for practicing the invention include formic acid, ethanol, isopropanol, cycloheptatriene, derivatives of cycloheptatriene, heterocylic compounds, polymers, sugars, and the like.

A specific group of reducing agents are sodium hydrosulfite, hydroxymethanesulfinic acid, sodium hydrosulfate, potassium hydrosulfate, trialkyl phosphates, derivatives of phosphoric acid, polyacrylates, polyglycols, glucose, fructose, aldoses, glutathione, baker's yeast, and the like.

Complexing agents useful for practicing the invention include unidentates, bidentates, and poly dentates, and can contain anionic or cationic moieties. A specific group of complexing agents are ethylene diamine, ethylenediaminetetraacetic acid (EDTA), and betaine hydrocholoride. The most preferred are EDTA and betaine hydrochloride, and derivatives or salts thereof.

The compositions of the present invention can be formulated into products and compositions that can be used in oral are. Examples of suitable products include toothpastes, liquid pastes, gels, tooth powders, mouth wash, chewing gum and the like. Additional topical formulations suitable for oral care can be employed without departing from the scope of the present invention. The compositions of the present invention can be used in mouth pieces, compositions in dental trays, applied to dental floss or dentures, toothpicks or in combination with a formulation described herein.

In one embodiment, the reducing agents useful for practicing the invention are not anti-oxidants. Examples of anti-oxidants include vitamin C, vitamin E, benzoates and hydroxybenzoates, butylated hydroxytoluene (BHT), butylated hydroxyanisole (BHA), and other reducing phenols, derivatives of hydroxyquinoline, polymerized 2,2,4-trimethyl-1,2-dihydroquinoline and alkyl gallate such as dodecyl gallate, ethyl gallate, octyl gallate, propyl gallate. The acid form of vitamin C can be considered an anti-oxidant as well as its monovalent metal salts, with the sodium salt being widely available. Also, the esters of ascorbic acid may be used as anti-oxidants, for example the palmitic acid ester: ascorbyl palmitate. In addition, the use of enzymes such as, for example, cysteine proteinase, is not desired.

In another aspect, the invention relates a method to stabilize, in solution, using a suitable complexing agent, the reduced form of compounds believed to cause tooth stains.

The invention provides a method to make compounds that cause stains in teeth water soluble under conditions similar to normal oral care. Once the compounds that cause tooth stains are made water soluble, they can be easily removed, e.g., washed away with a rinse.

The invention will be illustrated by the following non-limiting examples. Vat and reactive phthalocyanine-based dyes were used in order to test the invention because they have some similarity in structure to the porphyrin-type compounds believed to cause tooth stains. The examples provided herein are not intended in a limiting, exhaustive, or exclusive sense.

### EXAMPLES

Materials

Betaine hydrochloride, dodecyl sulfate-sodium salt, ethylenediaminetetra-acetic acid-disodium salt, hydroxyapatite, hydroxymethanesulfinic acid-sodium salt, poly (ethylene glycol), poly (acrylic acid), sodium carbonate, sodium hydrosulfite, sodium hydroxide and cetylpyridinium chloride were obtained from Sigma-Aldrich, St. Louis. Sodium chloride from Alfa Aesar, Massachusetts, and tartaric acid from Fisher Scientific Company, New Jersey, were also used.

Hydroxyapatite was selected as a substrate to determine the stain removal effectiveness of the compositions of the present subject matter. Two forms of the hydroxyapatite (calcium phosphate hydroxide) were used in the examples. The first form, the as-received hydroxyapatite powder, was used as directly received from the vendor. In the second form, the hydroxyapatite powder was pressed into pellets of about 1.9 cm diameter and sintered in air at about 1100° C. for 2 hours. The density of the sintered specimen was about 65% of the theoretical density (3.156 g/cm³). These sintered hydroxyapatite pellets were then used in the examples.

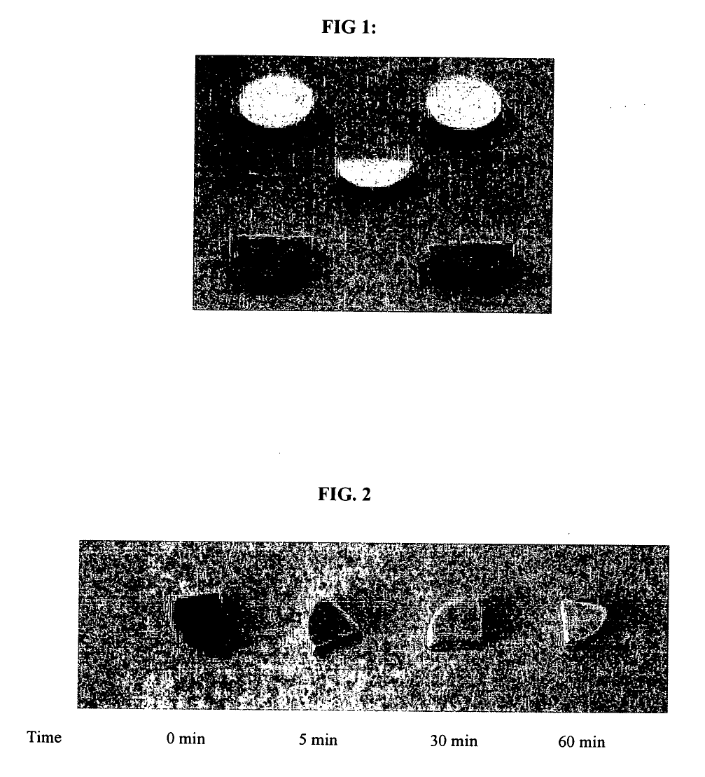

Both the powder and sintered pellet forms of hydroxyapatite are stained with dyes and their removal is studied using the non-oxidative compositions of the invention. FIG. 1 compares dye stained hydroxyapatite pellets to unstained pellets.

### Example 1

### Dye-staining Hydroxyapatite Pellets

For staining the hydroxyapatite with a vat dye, about 1.2 wt % dye solution [50 ml] was placed in a flask together with sintered hydroxyapatite pellets. To the flask, 5 ml each of sodium hydroxide [20 g/l ] and sodium hydrosulfite [20 g/l] solutions were added and the staining was carried out at 58° C. for about 8 hrs. During staining, an additional 2 ml of sodium hydrosulfite solution [20 g/l] was added after every 1 hr interval. After 8 hrs of staining, the flask was cooled to room temperature, the solution was discarded, and the stained pellets were washed with water until the surface residue was removed. The pellets were then dried at 50° C.

For staining with a reactive dye, about 2 wt % dye solution [50 ml], 5 ml sodium carbonate [20 g/l], 5 ml sodium hydroxide [20 g/l] and 2 g sodium chloride were placed in a flask together with sintered hydroxyapatite pellets. The contents in the flask were then heated to 45° C. and maintained for 24 hours. Finally, the contents were cooled to room temperature, and the samples were removed, washed, and then dried at 50° C.

### Example 2

### Stain Removal

Various solutions suitable for stain removal were prepared as described in the present application and recited in Table 1. Sodium hydrosulfite, which is the reducing agent present in Solution A, has limited stability. Therefore, hydroxymethanesulfinic acid-monosodium salt was also used in Solution A. Together, these compounds provide stronger reducing conditions. Cetyl pyridinium chloride is added to Solution A as a surfactant Solution A: A mixture sodium hydroxide, sodium hydrosulfite, cetylpyridinium chloride and hydroxymethanesulfinic acid—monosodium salt (See Table 1), was contacted with vat dye-stained hydroxyapatite samples. Solution A was maintained at 30-35° C. and occasionally shaken. The vat dye-stained hydroxyapatite samples in Solution A were removed after a few minutes, washed, and then dried at 50° C.

Solution B: A mixture of sodium hydroxide, sodium hydrosulfite, cetylpyridinium chloride, hydroxymethanesulfinic acid—monosodium salt, and tartaric acid (See Table 1), was contacted with reactive dye-stained hydroxyapatite samples at 30-35° C. for 5-10 min with intermittent shaking. After contacting for a few minutes, the pellets were washed with water and dried at 50° C.

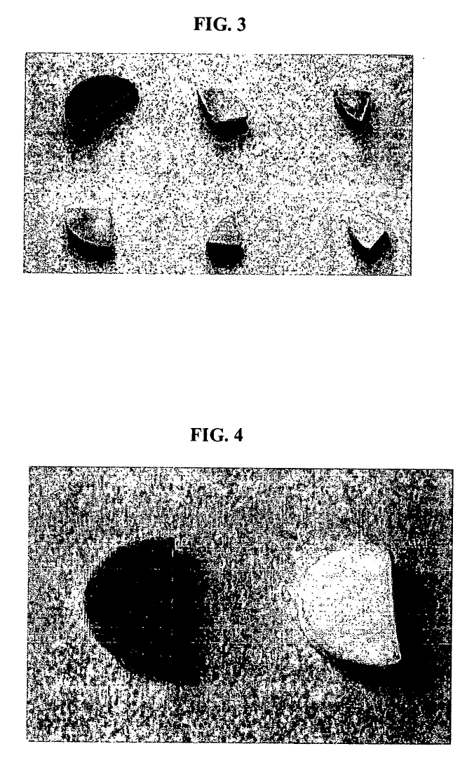

FIG. 2 illustrates vat dye-stain removal from hydroxyapatite pellets at times, 0 minutes; 5 minutes; 30 minutes; and 60 minutes; in Solution A. FIG. 3 also illustrates vat dye-stain removal from hydroxyapatite pellets in Solution A.

FIG. 4 illustrates reactive dye-stained pellet samples before and after stain removal using Solution B. The pellet samples were contacted with Solution B at 30-35° C. for 5-10 minutes and the reactive dye stains were almost completely removed.

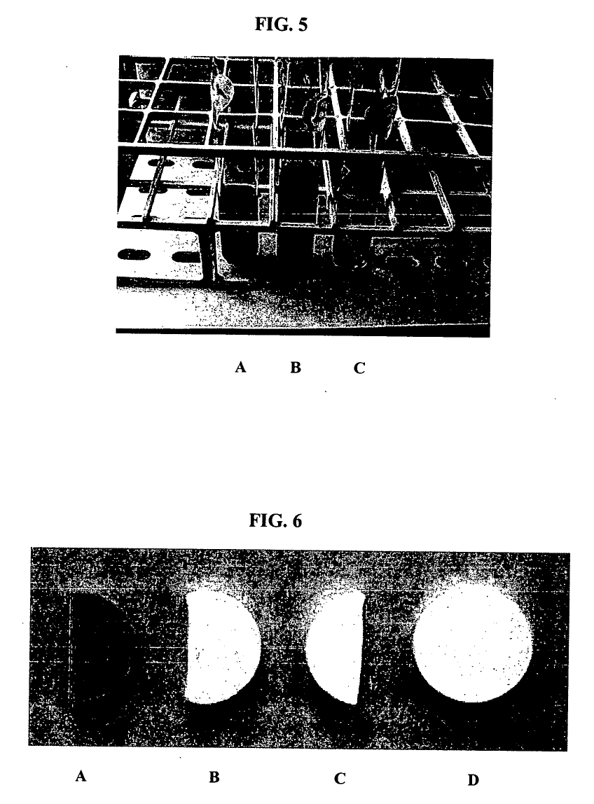

A reactive dye solution can change color in the presence of Solution A. In FIG. 5, the effect of Solution A in modifying stains is illustrated. When Solution A was added to a water soluble reactive dye solution, a change in color was observed instantly (See FIGS. 5B and 5C). This indicates that the chromophores in the reactive dye have been modified. The discoloration of a reactive dye solution is illustrated in FIG. 5.

TABLE 1
Description of various stain removal solutions prepared and used.

| Stain | | | | |
|---|---|---|---|---|
| removal | | | Quantity, | Stains |
| solution | Chemicals | Formula | g | removed |
| Solution A | Sodium hydroxide | NaOH | 0.1 | Vat Dyes |
| | Cetyl pyridinium chloride | C₂₁H₃₈NCl | 0.05 | |
| | Hydroxymethane | HOCH₂SO₂Na.2H₂O | 0.6 | |
| | sulfinic acid, salt | | | |
| | Sodium hydrosulfite | Na₂S₂O₄ | 0.3 | |
| | Water | H₂O | 25 | |
| Solution B | Sodium hydroxide | NaOH | 0.1 | Reactive Dyes |
| | Cetyl pyridinium chloride | C₂₁H₃₈NCl | 0.05 | |
| | Hydroxymethane | HOCH₂SO₂Na.2H₂O | 0.6 | |
| | sulfinic acid, salt | | | |
| | Sodium hydrosulfite | Na₂S₂O₄ | 0.3 | |
| | Tartaric acid | HOOC(CHOH)₂COOH | 0.05 | |
| | Water | H₂O | 25 | |
| Solution C | Sodium hydroxide | NaOH | 0.1 | Discoloration |
| | Hydroxymethane | HOCH₂SO₂Na.2H₂O | 0.6 | |
| | sulfinic acid, salt | | | |
| | Sodium hydrosulfite | Na₂S₂O₄ | 0.3 | |
| | Water | H₂O | 25 | |
| Solution D | Sodium hydroxide | NaOH | 0.1 | Discoloration |
| | Hydroxymethane | HOCH₂SO₂Na.2H₂O | 0.6 | |
| | sulfinic acid, salt | | | |
| | Sodium hydrosulfite | Na₂S₂O₄ | 0.3 | |
| | EDTA Di-sodium salt | C₁₀H₁₄N₂O₈Na₂.2H₂O | 0.05 | |
| | Water | H₂O | 25 | |
| Formulation-I | Hydroxymethane | HOCH₂SO₂Na.2H₂O | 1.0 | Tea and |
| | sulfinic acid, salt | | | coffee |
| | Sodium hydrosulfite | Na₂S₂O₄ | 0.6 | |
| | Betaine hydrochloride | (CH₃)₃NClCH₂CO₂H | 0.7 | |
| | Dodecyl sulphate, salt | C₁₂H₂₅O₄S.Na | 0.04 | |
| | Poly (acrylic acid) | H(OCH₂CH₂)ₙOH | 0.4 | |
| | Poly (ethylene glycol) | [—CH₂CH(CO₂Na)—]ₙ | 20 ml | |
| | Water | H₂O | 30 | |

### Example 3

### Addition of Complexing Agents

For this example, Solution A and Solution B are modified by adding EDTA-disodium salt. These modified solutions are then used to verify the degree of stain removal. Reactive blue 21, which contains Cu in phthalocyanine, was used to stain hydroxyapatite pellets. These stained pellets (FIG. 6A) were treated with Solution B (FIG. 6B) or Solution A (FIG. 6C) containing EDTA disodium salt at 32° C. for 3 cycles, each lasting for 5 min. The whiteness of this pellet was comparable in color to the original unstained hydroxyapatite pellet (FIG. 6D).

### Example 4

### Protocols for Staining with Tea or Coffee

Protocols for staining hydroxyapatite with tea or coffee: Hydroxyapatite as-received powder and pellets were stained with tea or coffee according to Protocols I and II. Stepwise procedures for these protocols are elaborated below.

Protocol I: Staining hydroxyapatite as-received powder with tea or coffee

Step I: Prepare tea solution in water
   Prepare tea by adding 50 g of tea to about 400 ml preheated water;
   Let the water continue to heat until boil and boil for 10 min;
   Remove the tea from the heat and cool to room temperature.
Step II: Stain hydroxyapatite powder with tea solution
   Add 25 g of as-received hydroxyapatite powder to 100 ml of tea solution as prepared in Step I;
   Adjust pH to about 8.0;
   Maintain tea solution containing hydroxyapatite powder for 2 days at 32° C. and cool it to room temperature;
   Decant the tea solution, wash, and centrifuge until washing is clear;
   Dry stained powder at 65° C. until constant weight.
      Protocol-II: Staining hydroxyapatite pellet with tea or coffee
Step I: Preparing hydroxyapaptite pellet
   Weigh about 20 g of hydroxyapaptite powder and wet-mill in 40 ml of ethanol for 2 hr using alumina media;
   Drive off ethanol completely at 60° C. under vacuum;
   Press powder into pellets.
Step II: Sinter hydroxyapatite pellet
   Sinter hydroxyapatite pellet at 1100° C. for 2 hr.
Step III: Prepare tea solution in water
   Prepare tea by adding 3 tea bags (or about 15-20 g) to about 150 ml preheated water
   Once the bags are added, let the water continue to heat until boil and boil for 10 minutes
   Remove the tea from the heat and cool to room temperature.
Step IV: Stain hydroxyapatite pellet with tea solution
   Add previously prepared hydroxyapatite pellets to 100 ml of tea solution as prepared in Step III
   Adjust pH to about 8.0
   Place hydroxyapatite pellet in tea solution for 2 days at 32° C. and cool to room temperature
   Decant the tea solution and dry the pellet at 65° C. until constant weight.

### Example 5

### Modification of Coffee Stains

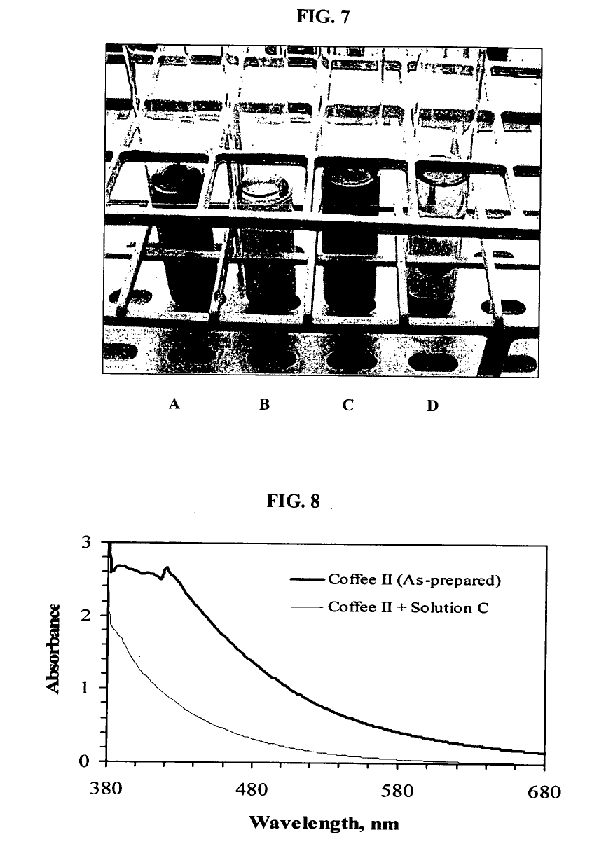

Two brands of coffee (Folgers Instant (Coffee I) and Eight O'clock Filter (Coffee II)) were chosen and a water-based solution of each was prepared in a fashion similar to Protocol I in Example 4 (FIGS. 7A and 7C, respectively). The initial pH was adjusted to 8.0, and to 10 ml of each coffee solution, about 0.5-1.0 ml of Solution B (to Coffee I) or Solution C (to Coffee II) was added at room temperature. After addition of Solution B to the Coffee I solution (FIG. 7B) or solution C to the Coffee II solution (FIG. 7D), discoloration was observed almost instantaneously. Although the solutions became slightly turbid, the discoloration indicates that the chromophores present in coffee are modified.

FIG. 8 illustrates a comparison the UV-visible spectra of Coffee II as-prepared with Coffee II+Solution C. After the addition of Solution C to the Coffee II solution, discoloration was observed. This figure provides spectroscopic evidence of the modification of the chromophores present in the Coffee II solution.

### Example 6

### Modification of Tea Stains

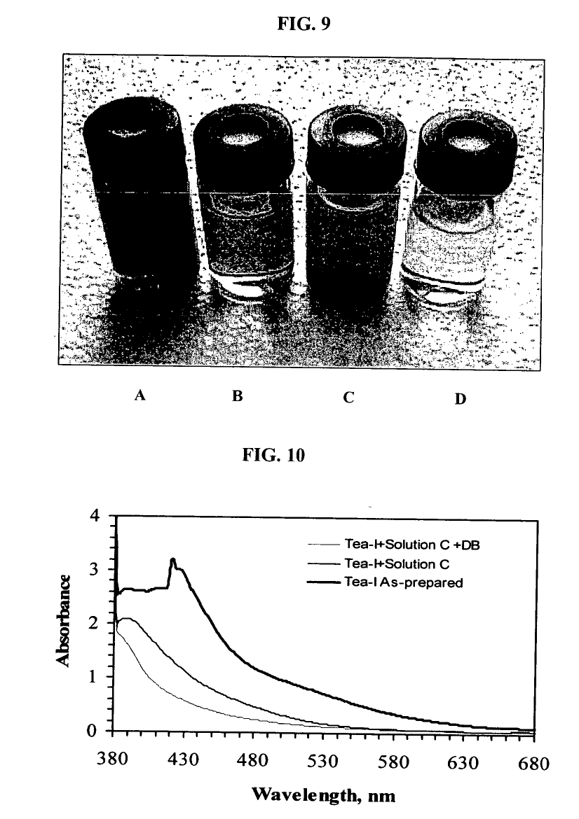

Two commercial brands of tea (Kroger tea bags (Tea-I) and BrookBond Red Label powder (Tea-II)) were obtained, and their solutions were prepared as per the procedure outlined in Example 4. To 10 ml of Tea-I solution, 1 ml of Solution C was added, and discoloration of the solution was observed within about 2 min. Upon further addition of a small amount of domiphen bromide (DB) to the Tea-I, the solution became turbid (as shown in FIG. 9C). When the turbid solution was filtered, the filtrate was almost clear, and discoloration was significant (FIG. 9D). The Tea-I solution as-prepared (FIG. 9A), and Tea-I+Solution C without (FIG. 9B) and with (FIG. 9C) addition of DB, are shown in FIG. 9.

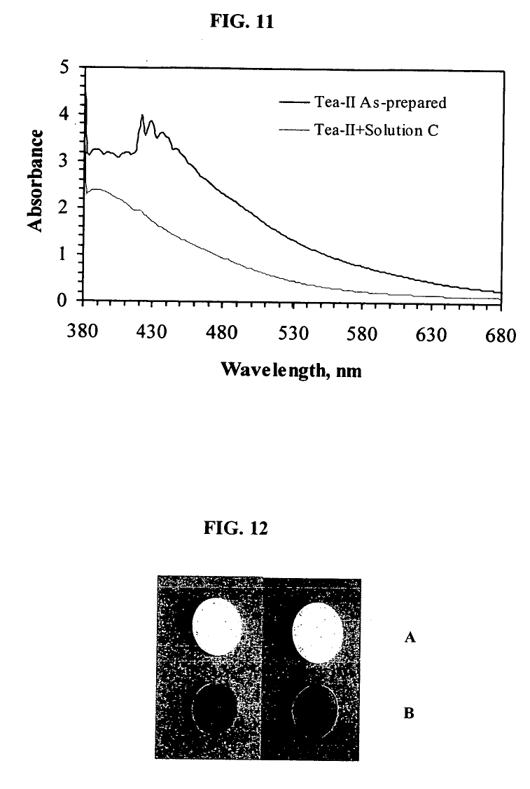

FIG. 10 shows the UV-visible spectra for Tea-I as-prepared, Tea-I+Solution C, and Tea-I+Solution C+DB. The absorbance value for the filtrate (as obtained after filtering a solution of Tea-I+Solution C+DB) is much lower than the value for the Tea-I as-prepared. FIG. 11 shows UV-visible spectra of Tea-II as-prepared and Tea-II+Solution C. These spectra indicate that modification of the chromophores occurred.

### Example 7

### Removal of Tea Stains

In FIG. 12, hydroxyapatite pellets stained with Tea-II (BrookBond Red label, FIG. 12B) are compared to unstained pellets (FIG. 12A). The Tea-II stained pellets were contacted with Solution C at 32° C. in 5 cycles, each lasting 5 min. The pellets were removed and washed thoroughly with water and dried at 50° C. until constant weight. They were then compared with Tea-II-stained hydroxyapatite pellets originally prepared, and the level of stain removal was assessed visually.

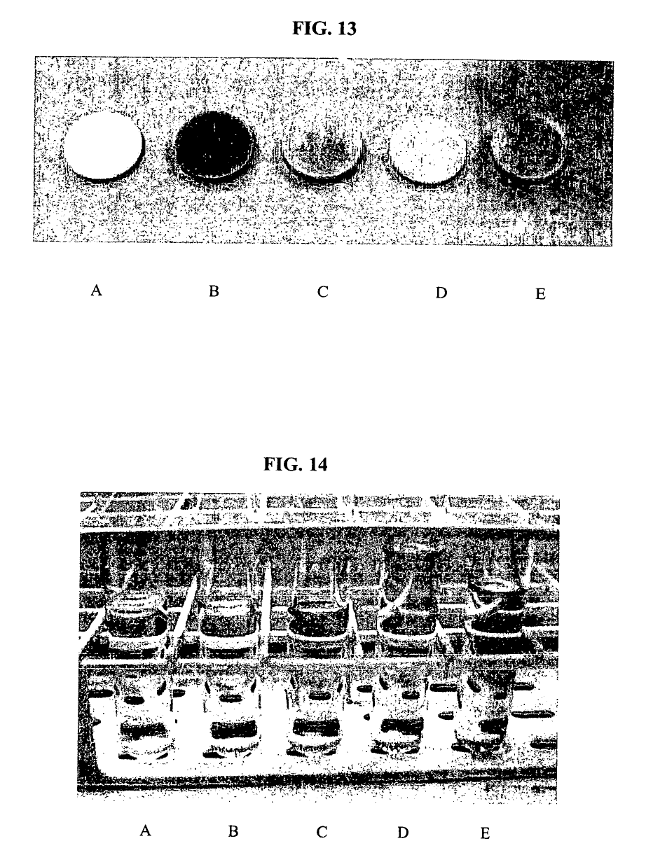

For a comparison, a Tea-II stained pellet was also contacted with a 1% H₂O₂ solution at 32° C. for 5 cycles of 5 min each. FIG. 13 shows the extent of stain removal obtained. The FIG. 13 legend is as follows:
FIG. 13A: As sintered Hydroxyapatite pellet;
FIG. 13B: Tea-II stained pellet;
FIG. 13C: Pellet after 5 minutes in Solution C;
FIG. 13D: Pellet after 50 cycles in Solution C, then EDTA; and
FIG. 13E: Pellet after treating with 1% H₂O₂ in 5 cycles.

After 5 cycles, the extent of Tea-II stain removal using Solution C was comparable with the one obtained using 1% H₂O₂. FIG. 14 shows the change in color of Solution C obtained after contact with a stained pellet for each 5-minute cycle. FIG. 14 illustrates that after each cycle of 5 min, Tea-II stain (color) was gradually extracted in the contacting solution. The FIG. 14 legend is as follows:
FIG. 14A: Pellets treated for 1 Cycle;
FIG. 14B: Pellets treated for 2 Cycles;.
FIG. 14C: Pellets treated for 3 Cycles;
FIG. 14D: Pellets treated for 4 Cycles; and
FIG. 14E: Pellets treated for 5 Cycles.

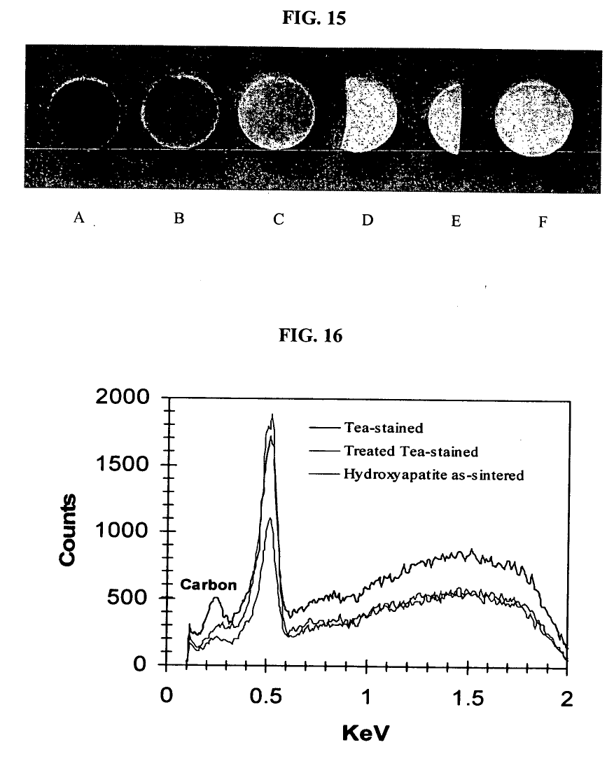

The results obtained after 50 contacting cycles of 5 min each (see FIG. 15) indicated that no more extraction of Tea-II stain (color) was possible. However, the whiteness of the pellet after 50 contacting cycles was not as close to the as-sintered unstained hydroxyapatite pellet originally prepared. Therefore, at the end of 50 contacting cycles, the pellet was removed and washed with water, and then contacted with a dilute EDTA solution for 5 minutes. Slightly more whitening was observed with this approach.

In addition, a comparison of the removal of a Tea-II stain from pellets with Solution C, followed by EDTA, and Solution D (Solution C and EDTA di-sodium salt) was conducted. (The EDTA was directly added to Solution C, to prepare Solution D). Tea-II stained pellets were contacted with Solution D for 5 cycles, each lasting 5 minutes. As seen in FIG. 15, the Tea-II stained pellets were much whiter as compared with sequential treatment of Solution C followed by EDTA solution. The FIG. 15 legend is as follows:
FIG. 15A: Tea-II stained pellet;
FIG. 15B: Pellet after 5 cycles in Solution C;
FIG. 15C: Pellet after 50 cycles in Solution C;
FIG. 15D: Pellet after 50 cycles in Solution C, followed by EDTA;
FIG. 15F: Pellet after 5 cycles in Solution D; and
FIG. 15E: Pellet unstained as-sintered.

FIG. 16 illustrates the energy-dispersive X-ray spectra of the as-sintered hydroxyapatite pellet, Tea-II stained hydroxyapatite pellet, and hydroxyapatite pellet first contacted with 50 cycles in Solution C followed by EDTA. The peak corresponding to carbon from tea (about 0.25 keV) is much reduced for the pellet first contacted with 50 cycles in Solution C followed by EDTA wash as compared with the Tea-II stained pellet originally prepared. This suggests that treatment of a Tea-II stained pellet with Solution C followed by EDTA has resulted in carbon removal, and hence whitening.

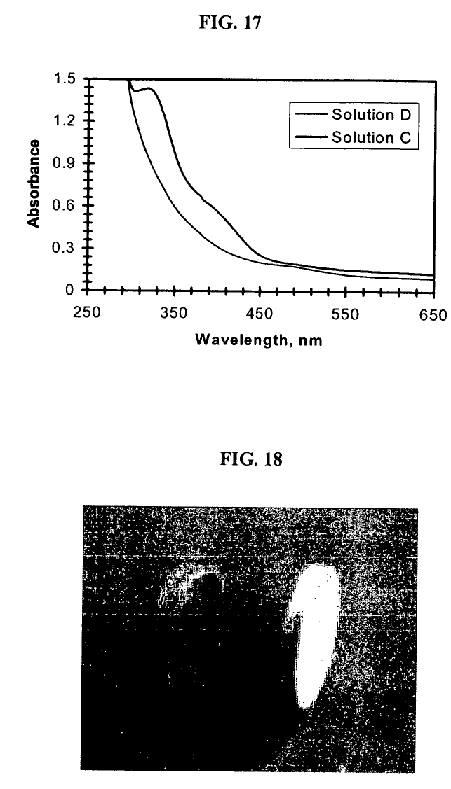

FIG. 17 illustrates the differences in the UV-visible spectra of Solution C and Solution D, obtained after contacting with Tea-II stained pellets. The only difference between Solution C and Solution D is that the latter contains EDTA. The different spectral absorbance is believed to be from color components extracted versus those extracted and complexed. Thus, FIG. 17 illustrates the role of EDTA in complexing with the chromophore containing compounds.

### Example 8

### Removal of Tea Stains

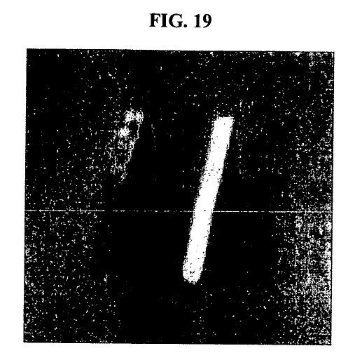

FIG. 18 illustrates stain removal from the external surfaces of a Tea II stained hydroxyapatite pellet contacted with Solution D. FIG. 18 shows that contacting with the Solution D, whitening of the external surfaces of the pellet has occurred. FIG. 19 illustrates the internal surfaces of the as-stained hydroxyapatite pellet and the Solution D treated pellet. Whitening of the internal surfaces is also observed.

### Example 9

### Removal of Tea Stains

This example for the removal of tea or coffee stains from hydroxyapatite powder is divided into two parts. Part 1 discusses the stepwise procedure to prepare a whitening solution, which is termed Formulation I. Part 2 discusses stain removal from powder using Formulation I.

Part 1: Preparation of Formulation I (see Table I for ingredient list)

Step 1: Prepare a solution containing 0.6 g of sodium hydrosulfite, 1.0 g of hydroxymethane sulfinic acid-sodium salt in about 20 ml of distilled water. Add this solution slowly to a beaker containing about 20 ml of poly (ethylene glycol) at room temperature. The pH of this solution is about 7.5. If the pH is acidic, then make it slightly alkaline by using a sodium hydroxide solution. Label this solution as Solution X.

Step 2: In another beaker, take 10 ml of distilled water and add 0.7 g of betaine hydrochloride. Stir it well to dissolve it completely. Then, add 0.04 g of dodecyl sulfate, sodium salt, and allow it to dissolve completely under occasional shaking. Label this solution as Solution Y.

Step 3: Add slowly Solution Y into Solution X under gentle stirring. Next, weigh about 0.4 g of poly (acrylic acid) and add it drop wise into the solution mixture of X and Y, and gently stir the solution mixture until it becomes clear.

The clear polymeric solution obtained in Step 3 is designated as Formulation I. This liquid formulation can be used for tea or coffee stain removal from hydroxyapatite powder or pellet.

Part 2: Procedure for stain removal using Formulation I

Tea or coffee stained hydroxyapatite powder, 0.5 g, and is added to a beaker containing about 20 ml of freshly prepared Formulation I solution. The solution containing the stained hydroxyapatite powder is heated to about 40° C and stirred continuously. The solution is maintained at about 40° C. for about 30 min under constant stirring and then centrifuged, decanted and the solution discarded. The stain removal treatment is repeated on the treated-wet-powder (without giving water wash) for six similar cycles using 20 ml of freshly prepared solution of Formulation I, each at a time.

After six cycles of stain removal treatment, wash the treated powder with 200 ml of water five times using 35-40 ml each at a time for about 5-10 min. The treated and washed wet hydroxyapatite powder should be neutral to pH paper. Dry the treated and washed powder at about 40° C.

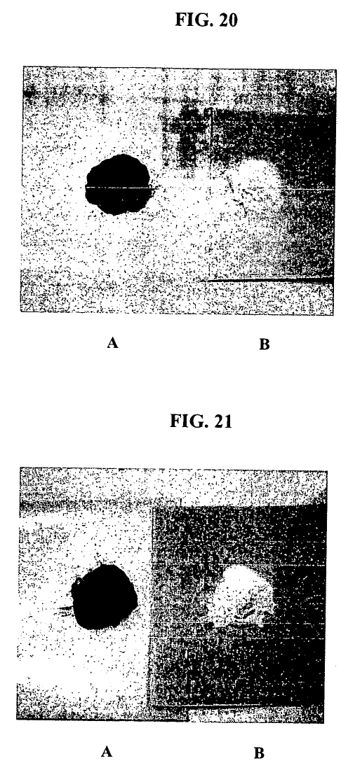

The results obtained in the present invention with tea and coffee stains and their removal from hydroxyapatite powder are illustrated in FIGS. 20, 21 and 22. FIG. 20A and 20B illustrate the whitening effect of Formulation I on tea-stained hydroxyapatite powder. FIG. 21 A and 21B illustrate the whitening effect of Formulation I on coffee-stained hydroxyapatite powder.

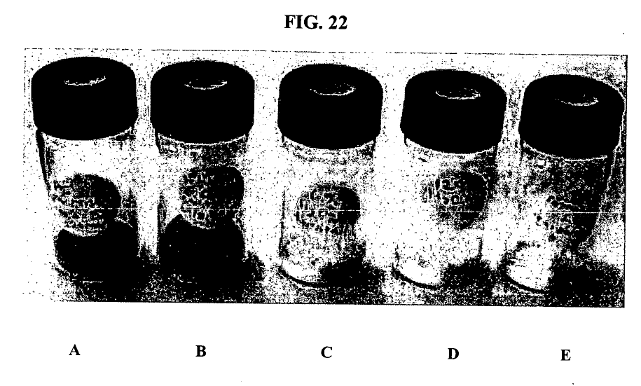

The whitening achieved in the case of tea-stained hydroxyapatite powder using Formulation-I was compared with the whitening achieved using an oxidizing agent such as H₂O₂. The results are shown in FIG. 22 and indicate that significant removal of tea stains is achieved using Formulation I. Similar levels of whitening were achieved on treating coffee-stained hydroxyapatite powder with Formulation I. The FIG. 22 legend is as follows:
FIG. 22A: Tea-stained powder;
FIG. 22B: Powder treated with Solution C;
FIG. 22C: Powder treated with 6% H₂O₂;
FIG. 22D: Powder treated with 12% H₂O₂; and
FIG. 22E: Powder treated with Formulation-I.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A non-oxidative composition for removing or diminishing tooth stains comprising a reducing agent and optionally a complexing agent.

2. The composition of claim 1 wherein the reducing agent is selected from the group consisting of bisulfites, hydrosulphites, hydrosulfates, sulphurous acid (H₂SO₃), phosphorus, phosphates, phosphites, phosphoric acid, formic acid, ethanol, isopropanol, cycloheptatriene, derivatives of cycloheptatriene, heterocylic compounds, polymers, sugars, and salts thereof.

3. The composition of claim 1 wherein the reducing agent is selected from the group consisting of sodium hydrosulfite, hydroxymethanesulfinic acid, sodium hydrosulfate, potassium hydrosulfate, trialkyl phosphates, derivatives of phosphoric acid, polyacrylates, polyglycols, glucose, fructose, aldoses, glutathione, baker's yeast, and salts thereof.

4. The composition of claim 1 wherein the complexing agent is selected from the group consisting of unidentates, bidentates, polydentates, and related compounds that contain anionic or cationic moieties.

5. The composition of claim 1 which is useful for removing or diminishing stains in teeth.

6. The composition of claim 5 which is in the form of an ointment, paste or gel.

7. The composition of claim 5 wherein the composition is applied to the teeth via a dental tray.

8. The composition of claim 5 wherein the composition is applied to dentures.

9. The composition of claim 5 wherein the composition is chewing gum.

10. The composition of claim 3 wherein the reducing agent is sodium hydrosulfite.

11. The composition of claim 3 wherein the reducing agent is a sodium salt of sulphurous acid (H₂SO₃).

12. The composition of claim 4 wherein the complexing agent is ethylenediaminetetraacetic acid (EDTA); or a salt thereof.

13. The composition of claim 4 wherein the complexing agent is betaine hydrochloride.

14. A non-oxidative process for removing or diminishing stains in teeth comprising contacting the teeth with an effective amount of the composition of claim 1.

15. The process according to claim 14 wherein a water insoluble tooth stain undergoes a reaction modification and solubilization in water to be removed from the tooth.

16. The process according to claim 15 wherein the solubilized stain is complexed with a suitable reducing agent and a suitable complexing agent and stabilized in water.

17. The process according to claim 16 wherein the reducing agent is selected from the group consisting of bisulfites, hydrosulphites, hydrosulfates, sulphurous acid (H₂SO₃), phosphorus, phosphates, phosphites, phosphoric acid, formic acid, ethanol, isopropanol, cycloheptatriene, derivatives of cycloheptatriene, heterocylic compounds, polymers, sugars, and salts thereof.

18. The process according to claim 16 wherein the reducing agent is selected from the group consisting of sodium hydrosulfite, hydroxymethanesulfinic acid, sodium hydrosulfate, potassium hydrosulfate, trialkyl phosphates, derivatives of phosphoric acid, polyacrylates, polyglycols, glucose, fructose, aldoses, glutathione, baker's yeast, and salts thereof.

19. The process according to claim 16 wherein the complexing agent is selected from the group consisting of unidentates, bidentates, polydentates, and related compounds that contain anionic or cationic moieties.

20. The process according to claim 16 wherein the reducing agent is sodium hydrosulfite.

21. The process according to claim 16 wherein the complexing agent is ethylenediaminetetraacetic acid (EDTA); or a salt thereof.

22. The process according to claim 16 wherein the complexing agent is betaine hydrochloride.

23. The non-oxidative process for removing or diminishing stains from a surface using a composition of claim 1.

24. The process according to claim 23 wherein the surface is tooth enamel.
